(19) United States
(12) Patent Application Publication
(10) Pub. No.: US 2007/0128294 A1
(43) Pub. Date: Jun. 7, 2007

(54) Treatment and prevention of liver adverse conditions using gallium

(76) Inventors: Louis R. Bucalo, Miami, FL (US); Sunil Sreedharan, South San Francisco, CA (US); Krishna P. Allamneni, Sunnyvale, CA (US); Lawrence R. Bernstein, Menlo Park, CA (US)

Correspondence Address: MORRISON & FOERSTER LLP, 755 PAGE MILL RD, PALO ALTO, CA, 94304-1018, US

(21) Appl. No.: 11/594,582

(22) Filed: Nov. 7, 2006

Related U.S. Application Data
(60) Provisional application No. 60/734,406, filed on Nov. 7, 2005.

(51) Int. Cl. A61K 38/16; A61K 33/24; A61K 31/28

(52) U.S. Cl. 424/617; 514/6; 514/492

(57) ABSTRACT

The invention provides methods and compositions for treating adverse conditions of the liver in an individual. A pharmaceutical composition including gallium, in the form of a coordination complex of gallium (III), a salt of gallium (III), an inorganic gallium (III) compound other than a gallium salt, or protein-bound gallium (III), together with a pharmaceutically acceptable carrier, is administered to the individual in an amount sufficient to provide a therapeutically or prophylactically effective serum gallium level.

### CROSS-REFERENCE TO RELATED APPLICATIONS

This application claims the benefit of U.S. provisional application No. 60/734,406, filed Nov. 7, 2005, the contents of which are hereby incorporated by reference in their entirety.

### TECHNICAL FIELD

This invention relates to gallium-containing compositions and their use in the treatment and prevention of adverse conditions of the liver.

### BACKGROUND

The liver is the body's largest internal organ, performing a multitude of functions essential to the maintenance of health. It plays vital roles in metabolism, digestion, immunity, and the regulation of blood chemistry. It also constitutes the body's main means of detoxifying and removing harmful substances from the blood. Although the liver has a remarkable ability to heal itself, and even to regenerate after injury, repeated exposure to agents that are toxic, infectious, or otherwise damaging can lead to a variety of liver diseases and disorders. These diseases and disorders, because of the liver's many crucial functions, are usually serious, and are commonly fatal.

The many known functions of the liver include detoxifying harmful substances derived from ingested food, beverages, and drugs; removing potentially harmful substances from the blood, such as toxic metals, antigens, microbial toxins, certain bacteria and viruses, and old or damaged erythrocytes; producing and regulating plasma for the lymphatic system; helping to regulate blood levels of glucose, fatty acids, phospholipids, many amino acids, many vitamins, iron, copper, blood clotting factors, cholesterol (including HDL and LDL), many hormones (including estrogens, androgens, and thyroid hormone), bilirubin, “acute phase” proteins (including complement and many cytokines), and water. One visible manifestation of liver damage is jaundice, a yellow coloration of the skin and eyes, which results from incomplete excretion of bilirubin. It is clear from this partial list of liver functions why damage to the liver can have profound effects on health.

Liver damage can result from exposure of the liver to toxins contained in, or derived from, food, beverages, drugs, the air, or infectious agents. The toxins may have been ingested, inhaled, absorbed through the skin, absorbed through mucous membranes, or derived from endogenous infections or other diseases. Damage can also result from, for example, radiation, heat, or physical trauma. A common cause of damage is exposure to alcohol (usually ethanol) from the ingestion of alcoholic beverages. Damage can be acute, from exposure to high alcohol concentrations, or cumulative, from exposure to lower alcohol concentrations over an extended period of time. Another common cause of liver damage is viral hepatitis, particularly hepatitis B or C. Over time, liver damage from such causes can lead to liver cirrhosis, which is the permanent destruction of liver tissue (with normal liver tissue becoming replaced by connective tissue), and the resultant loss of liver functions. The loss of liver functions leads to a variety of health problems, including toxemia and the consequent damage to other organs, and is commonly fatal.

While there are a number of medications available that are intended to treat particular causes of adverse liver conditions, they are generally specific to particular damaging agents, and therefore do not provide broad protection for the liver. For example, bacterial liver infections can commonly be treated successfully with particular antibiotics. There are, however, no consistently effective treatments for many serious liver diseases, including hepatitis B, hepatitis C, and cirrhosis

Thus, the need exists for an effective treatment for a wide variety of adverse liver conditions, especially for the many such conditions that do not now have any or adequate methods of treatment. Additionally, a need exists for a more general treatment of an adverse liver condition that can be used prior to full diagnosis of what the condition is. Further, a need exists for a preventive treatment to decrease or eliminate adverse liver conditions that result from future exposure to causative agents.

### BRIEF SUMMARY OF THE INVENTION

The invention provides methods, compositions, and kits for treating, mitigating, or preventing adverse liver conditions in an individual.

In one aspect, the invention provides a method for treating, mitigating, or preventing an adverse condition of the liver, comprising administering to the individual a unit dose of a gallium-containing composition, wherein the unit dose comprises an amount of the gallium-containing composition sufficient to provide a therapeutically or prophylactically effective serum gallium level. In one embodiment, the invention provides a method for treating an adverse condition of the liver in an individual in need thereof, comprising administering to the individual a unit dose of a gallium-containing composition, wherein the unit dose comprises an amount of the gallium-containing composition sufficient to provide a therapeutically effective serum gallium level. In another embodiment, the invention provides a method for mitigating potential liver damage resulting from administration of a pharmacologically active agent or radiation therapy, or exposure of an individual to a toxic substance, comprising administering a unit dose of a gallium-containing composition before, during, or subsequent to administration of the pharmacologically active agent or radiation therapy, or exposure of the individual to the toxic substance, wherein the unit dose comprises an amount of the gallium-containing composition sufficient to provide a prophylactically effective serum gallium level.

In methods of the invention, a therapeutically or prophylactically effective serum gallium level is typically at least about 10 ng/mL. In other embodiments, the serum gallium level is at least about 25, 50, 100, 200, or 500 ng/mL. In other embodiments, the serum gallium level is about 10 to about 50 ng/mL, about 25 to about 100 ng/mL, about 100 to about 500 ng/mL, about 500 to about 1000 ng/mL, about 50 to about 10,000 ng/mL, about 100 to about 7,500 ng/mL, about 200 to about 5,000 ng/mL, or about 500 to about 2,000 ng/mL.

In various embodiments, the therapeutically or prophylactically effective serum gallium level is reached within at least about 1, 2, 6, 12, 24, 48, or 72 hours following administration of the gallium-containing composition to the individual. In some embodiments, the therapeutically or prophylactically effective serum gallium level is reached within about 1 to about 12, about 6 to about 12, about 12 to about 24, about 24 to about 48, or about 48 to about 72 hours.

In the methods described herein, the gallium-containing composition may comprise or consist essentially of a coordination complex of gallium (III), a salt of gallium (III), an inorganic gallium (III) compound other than a salt, or protein-bound gallium (III). A gallium-containing composition is generally formulated in a pharmaceutical composition, comprising the gallium-containing composition together with a pharmaceutically acceptable carrier.

In some embodiments, the gallium-containing composition comprises a salt of gallium (III), for example an inorganic salt, e.g., selected from gallium nitrate, gallium chloride, gallium carbonate, and gallium sulfate, or hydrated or solvated forms thereof, or combinations thereof.

In some embodiments, the gallium-containing composition comprises an inorganic compound of gallium (III) other than a gallium salt, e.g., selected from gallium oxide, gallium oxide hydroxide, or hydrated or solvated forms thereof, or combinations thereof.

In some embodiments, the gallium-containing composition comprises an organic salt, e.g., selected from gallium acetate, gallium tartrate, gallium citrate, gallium formate, gallium oxalate, gallium gluconate, gallium ascorbate, gallium palmitate, and gallium hydroxamate, or hydrated or solvated forms thereof, or combinations thereof.

In some embodiments, the gallium-containing composition comprises a coordination complex of gallium (III), for example a complex comprising three identical or non-identical bidentate ligands coordinated to a gallium center. In some embodiments, the gallium-containing composition comprises a coordination complex in the form of a neutral 3:1 (hydroxypyrone:gallium) complex in which each hydroxypyrone molecule is either unsubstituted or substituted with one, two, or three C₁-C₆ alkyl substituents. In some embodiments, each hydroxypyrone molecule is unsubstituted or substituted at the 2-, 5-, and/or 6-positions with a C₁-C₆ alkyl group, or combinations thereof. In some embodiments, each hydroxypyrone is selected from the group consisting of 3-hydroxy-4-pyrone, 3-hydroxy-2-methyl-4-pyrone, 3-hydroxy-2-ethyl-4-pyrone, and 3-hydroxy-6-methyl-4-pyrone. In one embodiment, each hydroxypyrone molecule is 3-hydroxy-2-methyl-4-pyrone. In another embodiment, each hydroxypyrone molecule is 3-hydroxy-2-ethyl-4-pyrone. In some embodiments, the gallium-containing composition comprises a coordination complex of gallium (III) wherein the ligands are of the formula Ar—O— wherein Ar is an aryl, heteroaryl, substituted aryl, or substituted heteroaryl group. In one embodiment, the complex comprises the anion of 8-hydroxyquinoline. In some embodiments, the ligands are selected from carboxylate ligands having the structure R—(CO)—O— where R is hydrocarbyl, substituted hydrocarbyl, heteroatom-containing hydrocarbyl, or substituted heteroatom-containing hydrocarbyl, and combinations thereof.

Adverse liver conditions which may be treated, mitigated, or prevented by the methods described herein include liver diseases, disorders, and other conditions that damage the liver. Examples of such conditions include liver disease caused by alcohol use, drug use, hepatotoxic medication, radiation, physical injury, exposure to a hepatotoxic substance, traumatic injury to the liver, or infection (including viral, bacterial, mycoplasmal, fungal, protozoan, parasitic, or helminthian infections). In some embodiments, the adverse condition of the liver comprises hypertrophy of the liver (hepatomegaly).

Examples of liver diseases, and of other diseases that can damage the liver, include hepatic steatosis, non-alcoholic steatohepatitis, primary biliary cirrhosis, biliary atresia, hemochromatosis, alpha-1-antitrypsin deficiency, type-1 glycogen storage disease, porphyria, tyrosinemia, Wilson's disease, autoimmune hepatitis, neonatal hepatitis, Reye's syndrome, sarcoidosis, cystic liver disease (including choledochal cysts, Caroli's syndrome, congenital hepatic fibrosis, and polycystic liver disease), inflammatory liver disease (e.g., primary sclerosing cholangitis), cystic fibrosis, tuberculosis, Byler's disease, and Niemann-Pick disease. In some embodiments, the liver disease comprises hepatitis (e.g., chronic hepatitis, acute hepatitis, lupoid hepatitis, autoimmune hepatitis, or viral hepatitis). In some embodiments, the liver disease comprises hepatitis caused by a virus selected from the group consisting of hepatitis A, hepatitis B, hepatitis C, hepatitis D, hepatitis E, hepatitis non A-E, cytomegalovirus, Epstein-Barr virus, and combinations thereof. In some embodiments, the liver disease comprises hepatitis caused by a bacterial infection, e.g., caused by a bacterium selected from the group consisting of leptospira, rickettsia, and streptococcus species, and combinations thereof, a mycoplasmal infection, a protistal infection, or a helminthian infection.

In one embodiment, the adverse liver condition is caused by a hepatotoxic medication, e.g., a prescription or non-prescription drug. In some embodiments, the hepatotoxic medication is selected from anti-inflammatory agents, lipid-lowering agents, immunosuppressant agents, antidiabetic agents, antibiotics, antifungal agents, retinoids, anticonvulsant agents, psychotropic agents, and hormones, and combinations thereof. In some embodiments, the hepatotoxic medication is selected from NSAIDs (e.g., acetaminophen), statins, nicotinic acid, acarbose, cyclosporine, pioglitazone, sulfonylureas, amoxicillin, clarithromycin, erythromycin, tetracycline, trolendomycin, isoniazid, nitrofurantoin, fluconazole, fluoxetine, itraconazole, ketoconazole, etretinate, phenytoin, valproic acid, bupropion, chlorpromazine, tricyclic antidepressants, tamoxifen, testosterone, halothane, methotrexate, pyrazinamide, cocaine, and combinations thereof.

In one embodiment, the adverse liver condition is caused by exposure of an individual to a toxic substance, such as, for example, an environmental pollutant, a halide-hydrocarbon, petroleum, a petroleum byproduct, a pesticide, a chemical compound used in manufacturing, an organic solvent, or combinations thereof. In some embodiments, the toxic substance is derived from a plant containing pyrrolizidine alkaloids, e.g., from the Asteraceae family (daisy), Boraginaceae family (borage), Teucrium chamedrys (germander), Larrea tridentate (chaparral), Acorus species, and Asarum species.

In methods of the invention, the gallium-containing composition may be administered in a single daily dose or in multiple doses, e.g., 2, 3, 4, or more doses, per day. Generally, when administered to a human, the gallium-containing composition is administered to provide a total daily amount of gallium of about 2 to about 800 mg/kg/day. In some embodiments, the total daily amount of gallium administered is about 2 to about 15, about 8 to about 40, about 15 to about 80, about 40 to about 160, about 150 to about 325, about 300 to about 550, about 500 to about 700, or about 600 to about 800 mg/kg/day. The gallium-containing composition may be administered orally or parenterally, e.g., intravenously, subcutaneously, intramuscularly, transdermally, transmucosally, by inhalation, or via an implanted reservoir. In one embodiment, the gallium-containing composition is administered orally in one or more oral dosage forms per day. In one embodiment, the gallium-containing composition is a complex of gallium (III) and 3-hydroxy-2-methyl-4-pyrone, and is administered orally once a day.

In some embodiments, the gallium-containing composition is administered in combination with a second active agent indicated for treatment of an adverse liver condition. In one embodiment, the adverse liver condition is hepatitis, and the second active agent is a cytokine, e.g., an interferon (for example, a-interferon), a nucleoside agent, or a combination thereof. In another embodiment, the adverse liver condition is bacterial hepatitis, and the second active agent is an antibacterial agent.

In another aspect, the invention provides a pharmaceutical composition for treatment of an adverse condition of the liver, comprising an amount of a gallium-containing composition to provide a therapeutically or prophylactically effective serum gallium level, and a therapeutically effective amount of a second agent indicated for treatment of the adverse liver condition.

In a further aspect, the invention provides a kit for treatment, mitigation, or prevention of an adverse condition of the liver, comprising at least one unit dose of a gallium-containing composition, wherein the unit dose comprises an amount of the gallium-containing composition sufficient to provide a therapeutically or prophylactically effective serum gallium level following administration of the composition to an individual. Kits may further provide packaging and/or instructions for use of the gallium-containing composition to treat or mitigate the adverse condition of the liver. In one embodiment, the kit comprises at least one oral dosage form comprising the gallium-containing composition formulated for oral administration. In some embodiments, the oral dosage form comprises a coordination complex in the form of a neutral 3:1 (hydroxypyrone:gallium) complex in which each hydroxypyrone molecule is either unsubstituted or substituted with one, two, or three C₁-C₆ alkyl substituents, for example, 3-hydroxy-4-pyrone, 3-hydroxy-2-methyl-4-pyrone, 3-hydroxy-2-ethyl-4-pyrone, or 3-hydroxy-6-methyl-4-pyrone. In one embodiment, each hydroxypyrone molecule is 3-hydroxy-2-methyl-4-pyrone. In another embodiment, each hydroxypyrone molecule is 3-hydroxy-2-ethyl-4-pyrone.

### BRIEF DESCRIPTION OF THE DRAWINGS

FIG. 1. Liver weights of normal rats (normal control), untreated adjuvant arthritic rats (disease control), and adjuvant arthritic rats treated for 21 days with 100 or 300 mg/kg/day oral gallium maltolate, or 0.1 mg/kg/day intraperitoneal dexamethasone. Indicated percentages express the reduction in liver weight relative to disease controls, with normal controls representing 100% reduction.

FIG. 2. Liver from untreated adjuvant arthritic disease control rat. FIG. 2A shows the view at 100-× magnification. An accentuated lobular pattern is observed, with centrilobular areas (outlined in black, with white arrows) stained paler than periportal areas (black arrow). FIG. 2B shows a higher magnification (400-×) view of periportal area, showing hypertrophy. Representative hepatocytes are outlined in black; these are enlarged relative to hepatocytes shown in FIG. 2C and FIG. 3. Black arrows identify liver macrophages (Kupffer cells) lining sinusoids. FIG. 2C shows a higher magnification (400-×) view of centrilobular area. Representative hepatocytes are outlined in black. Black arrows identify Kupffer cells lining sinusoids.

FIG. 3. Liver from adjuvant arthritic rat that had been treated with 300 mg/kg/day oral gallium maltolate for 21 days. FIG. 3A shows a view at 100× magnification. No accentuation of the lobular pattern is seen, with centrilobular areas (outlined in black) staining similarly to periportal areas (white arrow). FIG. 3B shows a higher magnification (400-×) view of periportal area. Representative hepatocytes are outlined in black. Black arrows identify Kupffer cells lining sinusoids. FIG. 3C shows a higher magnification (400-×) view of centrilobular area. Representative hepatocytes are outlined in black. Black arrows identify Kupffer cells lining sinusoids.

FIG. 4. Effect of gallium maltolate on alanine aminotransferase (ALT) activity in Con A-treated mice.

FIG. 5. Effect of gallium maltolate on liver centrilobular necrosis in Con A-treated mice.

### DETAILED DESCRIPTION

### Definitions

Unless otherwise indicated, the invention is not limited to specific synthetic methods, analogs, substituents, pharmaceutical formulations, formulation components, modes of administration, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a substituent” includes a single substituent as well as two or more substituents that may be the same or different, reference to “a compound” encompasses a combination or mixture of different compounds as well as a single compound, reference to “a pharmaceutically acceptable carrier” includes two or more such carriers as well as a single carrier, and the like.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred lower alkyl substituents contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage, or substituent in which one or more carbon atoms are replaced with an atom other than carbon, e.g., nitrogen, oxygen, sulfur, phosphorus, germanium, or silicon, typically nitrogen, oxygen or sulfur, preferably nitrogen or oxygen. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulthydryl, C₁-C₂₄ alkoxy, C₂-C₂₄ alkenyloxy, C₂-C₂₄ alkynyloxy, C₅-C₂₄ aryloxy, acyl (including C₂-C₂₄ alkylcarbonyl (—CO-alkyl) and C₆-C₂₄ arylcarbonyl (—CO-aryl)), acyloxy (—O-acyl), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N-(C₁-C₂₄ alkyl), N—(C₆-C₂₄ aryl)-substituted carbamoyl, thiocarbamoyl (—(CS)—NH₂), carbamido (—NH—(CO)—NH₂), cyano(—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₂-C₂₄ alkenyl (preferably C₂-C₁₈ alkenyl, more preferably C₂-C₁₂ alkenyl, most preferably C₂-C₆ alkenyl), C₂-C₂₄ alkynyl (preferably C₂-C₁₈ alkynyl, more preferably C₂-C₁₂ alkynyl, most preferably C₂-C₆ alkynyl), C₅-C₂₄ aryl (preferably C₅-C₁₄ aryl), C₆-C₂₄ alkaryl (preferably C₆-C₁₈ alkaryl), and C₆-C₂₄ aralkyl (preferably C₆-C₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present. Similarly, the phrase an “optionally present” bond as indicated by a dotted or dashed line - - - means that a bond may or may not be present.

When referring to a compound of the invention as an active agent, applicants intend the term “compound” or “active agent” to encompass not only the specified molecular entity but also its pharmaceutically acceptable, pharmacologically active analogs, including, but not limited to, salts, esters, amides, hydrates, solvates, prodrugs, conjugates, active metabolites, and other such derivatives, analogs, and related compounds.

The terms “treating” and “treatment” as used herein refer to causing a reduction in severity and/or frequency of symptoms, elimination of symptoms and/or underlying cause, prevention of the occurrence of symptoms and/or their underlying cause, and/or improvement or remediation of damage. Thus, “treating” a patient with a compound of the invention includes prevention of a particular disorder or adverse physiological event in a susceptible individual, as well as management of a clinically symptomatic individual to inhibit or cause regression of a disorder or disease. Treatment can include prophylaxis, therapy, or cure. For example, treatment of hepatitis encompasses chemoprevention in a patient susceptible to developing hepatitis (e.g., at a higher risk, as a result of genetic predisposition, environmental factors, predisposing diseases or disorders, or the like), as well as treatment of a patient with hepatitis by inhibiting, or causing regression of, the disease.

The term “effective amount” refers to the amount of a gallium-containing composition that provides gallium in a sufficient amount to render a desired treatment outcome. An effective amount may be comprised within one or more doses, i.e., a single dose or multiple doses may be required to achieve the desired treatment endpoint. A “therapeutically effective amount” refers to an amount of gallium-containing composition sufficient to produce a desired therapeutic outcome (e.g., reduction of severity of, or elimination of, an adverse liver condition). A “prophylactically effective amount” refers to an amount of gallium-containing composition sufficient to prevent or reduce severity of a future adverse liver condition when administered to an individual who is susceptible and/or who may develop an adverse liver condition, e.g., by virtue of exposure to a toxic substance.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

By “pharmaceutically acceptable” is meant a material that is not biologically or otherwise undesirable, i.e., the material may be incorporated into a pharmaceutical composition administered to a patient without causing any significant undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

An “individual” refers to a vertebrate, typically a mammal, commonly a human.

Methods of Use

Methods are provided for administration of a gallium-containing composition to an individual in need of treatment for, or prevention of, an adverse liver condition. Methods of the invention can be used to effect prophylaxis, therapy, or cure of an adverse condition of the liver. Methods include administration of one or more unit doses of a gallium-containing composition in a therapeutically or prophylactically effective amount. In methods of the invention, gallium-containing compositions are generally administered in a pharmaceutically acceptable carrier.

In methods of the invention, a gallium-containing composition is administered to an individual in an amount sufficient to provide a therapeutically or prophylactically effective serum gallium level for prevention or treatment of an adverse liver condition, such as liver hypertrophy resulting from exposure to one or more toxins. In one embodiment, the gallium-containing composition is administered in a unit dose that results in a gallium serum level, at about 24 hours following administration, of at least about 10 ng/mL. In various embodiments, a therapeutically or prophylactically effective serum level of gallium, at about 24 hours following administration, is at least any of about 10, 25, 50, 100, 200, or 500 ng/mL. In some embodiments of methods of the invention, the serum gallium level at Cₘₐₓ may be any of about 20 to about 50 ng/mL, about 25 to about 100 ng/mL, about 100 to about 500 ng/mL, about 500 to about 1,000 ng/mL, about 50 to about 10,000 ng/mL, about 100 to about 7,500 ng/mL, about 200 to about 5,000 ng/mL, or about 500 to about 2,000 ng/mL. In various embodiments, a unit dose of a gallium-containing composition providing a therapeutically or prophylactically effective amount of gallium results in a peak serum level of gallium of any of at least about 20, 50, 100, or 500, 1000, 2000, 3000, or 4000 ng/mL, with an upper limit of any of about 50, 100, 500, 1000, 2000, 3000, 4000, or 5000 ng/mL.

In methods of the invention, a therapeutically or prophylactically effective serum level is typically reached within about 1, 2, 6, 12, 24, 48, or 72 hours following administration of the gallium-containing composition to the individual. In some embodiments, the therapeutically or prophylactically effective serum gallium level is reached within about 1 to about 12 hours, about 6 to about 12 hours, about 12 to about 24 hours, about 24 to about 48 hours, or about 48 to about 72 hours.

A therapeutically or prophylactically effective dose may be administered as a single dose or in multiple doses per day, with the total daily dosage comprising a total dosage of about 2 to about 550 mg/kg/day gallium in a human individual. In various embodiments, methods of the invention comprise administering any of about 2, 3, 5, 6, 8, 16, 78, 160, 235, 315, 400, 470, 550, 600, 700, or 800 mg/kg/day gallium. In various embodiments, about 2 to about 15, about 10 to about 40, about 15 to about 80, about 40 to about 160, about 150 to about 325, about 300 to about 550, about 500 to about 700, or about 600 to about 800 mg/kg gallium is administered per day. In one embodiment, a once daily dosage of about 2 to about 15 mg/kg gallium is administered per day. In one embodiment, the method comprises administration of a pharmaceutical composition comprising gallium maltolate (a coordination complex of a trivalent gallium ion with three deprotonated maltol (2-methyl-3-hydroxy-4H-pyran-4-one)) groups at a dosage that results in an effective gallium serum level, at about 24 hours following administration, of at least about 10 ng/mL.

In some embodiments, two or more gallium-containing compositions may be co-administered. In some embodiments, one or more gallium-containing compositions are co-administered with one or more additional therapeutically beneficial substances, such as, for example, an interferon or an anti-microbial substance. Gallium-containing compositions

In accordance with methods of the invention as described herein, a gallium-containing composition can be administered that comprises, for example, a coordination complex of gallium (III), a salt of gallium (III), an inorganic compound of gallium (III) other than a salt, or protein-bound gallium (III). For administration to an individual, a pharmaceutical composition may be administered comprising a gallium-containing composition as described herein and a pharmaceutically acceptable carrier.

Gallium (III) coordination complexes are complexes that comprise a Ga(III) center coordinated to one or more ligands. Coordination complexes of gallium (III) include, without limitation, gallium (III) complexes of an N-heterocycle (such as tris (8-quinolinolato) gallium (III)), gallium (III) complexes with hydroxypyrones, including neutral 3:1 gallium complexes of a 3-hydroxy-4-pyrone (such as gallium maltolate), gallium complexes with hydroxypyridinones or substituted hydroxypyridinones, gallium porphyrins (such as gallium (III) protoporphyrin IX), pyridoxal isonicotinoyl hydrazone gallium (III), and gallium salt complexes of polyether acids. Such coordination complexes include, but are not limited to, those comprising three bidentate ligands or one tridentate ligand. Bidentate ligands are each coordinated to the gallium (III) center through two oxygen, nitrogen, or sulfur atoms; the two coordinating atoms may be the same or different. Similarly, tridentate ligands are coordinated to the gallium (III) center through three oxygen, nitrogen, or sulfur atoms; the three coordinating atoms may be the same or different. The coordinating ligands may all be the same or there may be a mixture of different ligands.

Bidentate ligands may be, for example, unsubstituted hydroxypyrone, or hydroxypyrone substituted at the 2-, 5-, and/or 6-positions with a C₁-C₆ alkyl group. In particular, bidentate ligands can be 2-substituted or 5-substituted hydroxypyrones, such as 3-hydroxy-2-methyl-4-pyrone (maltol) and 3-hydroxy-2-ethyl-4-pyrone (ethyl maltol). Other examples of bidentate ligands are unsubstituted hydroxypyridinones, or hydroxypyridinones substituted at the 2-, 5-, and/or 6-positions with a C₁-C₆ alkyl group. An example of a tridentate ligand is pyridoxal isonicotinoyl hydrazone.

Further, the ligands may be of the formula Ar—O—, wherein Ar is an aryl, heteroaryl, substituted aryl, or substituted heteroaryl group. For example, the Ar group may be an optionally substituted heteroaryl group such as the anion of 8-hydroxyquinoline.

The ligands also may be selected from carboxylate ligands having the structure R—(CO)—O—, where R is hydrocarbyl, a substituted hydrocarbyl, a heteroatom-containing hydrocarbyl, or a substituted heteroatom-containing hydrocarbyl.

In one embodiment, a gallium composition suitable for use in accordance with the methods of the invention comprises a gallium complex of a 3-hydroxy-4-pyrone, such as, for example, gallium maltolate. The synthesis of such complexes and preparations of the complexes in pharmaceutical formulations, have been described, for example, in U.S. Pat. Nos. 5,258,376, 5,574,027, 5,883,088, 5,968,922, 5,981,518, 5,998,397, 6,004,951, 6,048,851, and 6,087,354.

Gallium salts include both inorganic and organic salts. Examples of inorganic salts and related inorganic compounds include, but are not limited to, gallium chloride, gallium nitrate, gallium sulfate, gallium carbonate, and gallium phosphate. Hydrated and solvated forms of these salts are included. Examples of organic salts include, but are not limited to, gallium acetate, gallium citrate, gallium formate, gallium hydroxamate, gallium oxalate, gallium glutamate, gallium palmitate, and gallium tartrate, as well as their hydrated and solvated forms. Examples of inorganic gallium compounds other than gallium salts are gallium oxide and gallium oxide hydroxide, as well as their hydrated and solvated forms.

Other compositions suitable for use in the methods of the invention include peptides and proteins containing bound gallium. Examples of such compositions include gallium-lactoferrin and gallium-transferrin. In some embodiments, the protein is derived from the species to be treated. In some embodiments, protein-bound gallium-containing compositions are conjugated with one or more other active agents. An example of such a conjugate is gallium-transferrin-doxorubicin conjugate.

Adverse Conditions of the Liver

As used herein, “adverse liver condition” refers to a condition having a detrimental or potentially detrimental effect on the liver of an individual, often as a result of exposure to a toxin or pathogen. As used herein, “toxin” refers to a substance that causes or potentially may cause an adverse effect on the health of an individual who ingests, is administered, or is exposed to the substance. A “toxin” may be chemical or biological in origin. Many adverse liver conditions can be caused by prescription, non-prescription, and/or illicit drugs, natural or manufactured toxins, or a combination thereof. Some such toxins are released by manipulation of otherwise non-toxic materials, whereas some are byproducts of chemical reactions between otherwise non-harmful components.

Methods of the invention may reduce, alleviate, eliminate, or prevent at least one symptom of an adverse liver condition. Such symptoms include hypertrophy (enlargement) of the liver (hepatomegaly).

Examples of adverse liver conditions associated with exposure to drugs and/or toxins include liver disease caused by alcohol use or abuse, drug use or abuse, hepatotoxic medication, and/or exposure to other hepatotoxic substances. A large number of drugs have been found to be hepatotoxic. Examples of hepatotoxic drugs include, but are not limited to, some anti-inflammatory agents, lipid-lowering agents, immunosuppressant agents, antidiabetic agents, antibiotics, antifungal agents, retinoids, anticonvulsant agents, psychotropic agents, hormones, anticancer agents, protease inhibitors, amphetamines, proton pump inhibitors, and combinations thereof. Specific examples include some non-steroidal anti-inflammatory drugs (such as acetaminophen, aspirin, diclofenac, sulindac), statins, nicotinic acid, acarbose, pioglitazone, cyclosporine, sulfonylureas, amoxicillin, clarithromycin, erythromycin, tetracycline, trolendomycin, isoniazid, nitrofurantoin, fluconazole, fluoxetine, itraconazole, ketoconazole, etretinate, phenytoin, valproic acid, bupropion, chlorpromazine, tricyclic antidepressants, tamoxifen, testosterone, halothane, methotrexate, pyrazinamide, cocaine, and combinations thereof. Many of these drugs have increased hepatotoxicity when used with alcohol or with each other.

Other toxins that can cause adverse liver conditions include environmental pollutants such as petroleum and its volatile byproducts, pesticides, organic solvents, many heavy metals, and chemical compounds used in manufacturing.

The ingestion of certain aflatoxins, which are toxins than can occur in stored grains and other foods as the result of fungal growth, is also associated with the development of liver disease.

Native as well as transplanted flora can cause adverse liver conditions, especially when ground up and/or ingested. Examples include plants that contain pyrrolizidine alkaloids, such as those of the Asteraceae family (daisy), and the Boraginaceae family (borage). Other plants that can cause adverse liver conditions include many mushroom varieties, Echinacea (coneflower), Teucrium chamaedrys (germander), Larrea tridentate (chaparral), and Acorus and Asarum species.

Adverse liver conditions can also be caused by exposure to radiation or by physical trauma to the liver.

Examples of adverse conditions of the liver include alcoholic liver disease, primary biliary cirrhosis, primary sclerosing cholangitis, hemochromatosis, Wilson's disease, cystic liver diseases such as polycystic liver disease, congenital hepatic fibrosis, Caroli's syndrome, and inflammatory liver disease. Examples of inflammatory liver disease include autoimmune hepatitis, lupoid hepatitis, chronic hepatitis, and acute hepatitis.

Hepatitis can be caused by a viral infection, such as by hepatitis A virus, hepatitis B virus, hepatitis C virus, hepatitis D virus, hepatitis E virus, hepatitis non A-E virus, cytomegalovirus, Epstein-Barr virus, and combinations thereof. Bacterial and mycoplasmal infections can also cause hepatitis. Examples of bacteria that cause hepatitis in humans include Leptospira, Rickettsia, Streptococcus, and combinations thereof. Other organisms known to infect the liver and cause liver damage include protists such as Plasmodium spp. (which causes malaria), Leishmania donovani, Enterocytozoon bieneusi, and Entamoeba histolytica, and helminthes such as Schistosoma spp., Echinococcus spp., Clonorchis sinensis/Opisthorchis viverrini, and Fasciola hepatica.

Some individuals have a predisposition to adverse conditions of the liver, whether by intended future exposure to an agent that may cause adverse liver conditions, by a genetic predisposition for an adverse liver condition, by a predisposing disease or disorder, or by another cause. Preventive measures can be taken using methods of the invention to “mitigate,” i.e., prevent or limit the impact of the exposure or predisposition and/or one or more symptoms of the resulting liver condition. In some embodiments, one or more gallium-containing compounds may be administered prophylactically before, during, or after toxin exposure, to prevent or lessen the severity of a future adverse liver condition that may result from the toxin exposure. For example, in the case of future exposure to a causative agent, the causative agent can be administered in combination with an amount of gallium effective to provide a prophylactically effective serum gallium level, for example, simultaneously or within 1, 2, 6, 12, 24, 48, or 72 hours after administration of the causative agent. In some embodiments, a gallium-containing composition is administered in a dosage that results in a serum level of at least about 10 ng/mL for at least 24 hours.

An example of an adverse condition of the liver resulting from toxin exposure is hepatomegaly, or hypertrophy of the liver. Cirrhosis is one possible outcome of this condition, resulting from atrophy of the liver parenchyma and hypertrophy of the connective tissue. In some embodiments, an individual with hepatomegaly can be treated via methods of the invention by administering one or more gallium-containing compositions as described herein to diminish or alleviate the adverse condition. In one embodiment, a pharmaceutical composition comprising gallium maltolate is administered to an individual to diminish or alleviate hepatomegaly resulting from exposure to one or more toxic substances.

Modes of Administration

Administration of gallium-containing compounds in accordance with the methods of the invention may be via any route that provides a desired therapeutically or prophylactically effective serum level. Generally, one or more gallium-containing compositions is administered in a pharmaceutical composition that comprises a unit dose of the composition(s) and a pharmaceutically acceptable carrier. For example, administration may be oral or parenteral (e.g., intravenous, subcutaneous, intramuscular, transdermal, transmucosal (including buccal, nasal, rectal, sublingual, and vaginal), by inhalation, or via an implanted reservoir in a dosage form).

In some embodiments, a gallium containing composition, such as for example, a coordination complex of gallium (III), e.g., gallium maltolate, is administered orally. In some embodiments, the coordination complex is a complex of gallium (III) and 3-hydroxy-2-methyl-4-pyrone. In some embodiments, this complex is administered orally once per day to achieve and maintain a therapeutically or prophylactically effective serum level of gallium, for example, a serum level of at least 10 ng/mL.

Depending on the intended mode of administration, the pharmaceutical formulation may be a solid, semi-solid, or liquid, such as, for example, a tablet, a capsule, a caplet, a liquid, a suspension, an emulsion, a gel, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). For those compounds that are orally active, oral dosage forms are generally preferred, and include tablets, capsules, caplets, solutions, suspensions, and syrups, and may also comprise a plurality of granules, beads, powders, or pellets that may or may not be encapsulated. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose, and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also a preferred oral dosage form, in which case the active agent-containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

Oral dosage forms, whether tablets, capsules, caplets, or particulates, may, if desired, be formulated to provide gradual, sustained release of the active agent over an extended time period. Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The compounds of the invention may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the compounds may also be formulated as a depot preparation for controlled release of the active agent, preferably sustained release over an extended time period. These sustained release dosage forms are generally administered by implantation (e.g., subcutaneously or intramuscularly or by intramuscular injection).

Although the present compositions will generally be administered orally, parenterally, transdermally, or via an implanted depot, other modes of administration are suitable as well. For example, administration may be rectal or vaginal, preferably using a suppository that contains, in addition to the active agent, excipients such as a suppository wax. Formulations for nasal or sublingual administration are also prepared with standard excipients well known in the art. The pharmaceutical compositions of the invention may also be formulated for inhalation, e.g., as a solution in saline, as a dry powder, or as an aerosol.

Administration of Gallium in Combination with a Second Active Agent

In some embodiments, gallium is administered in combination with a second active agent that is indicated for treatment of the adverse liver condition. As an example, if the adverse liver condition is viral hepatitis, gallium can be administered with an interferon and/or a nucleoside agent. Administration can be simultaneous or in combination with gallium, or following appropriate regimens for each component, or administration of the gallium-containing composition and the second active agent may be sequential.

Methods for Mitigating Potential Liver Damage

In some instances, an identifiable cause of a potential adverse liver condition is known. The effects of such a cause can be mitigated by pretreating the individual with one or more gallium-containing compositions. Alternatively, the causative agent, such as a chemotherapeutic, can be administered together with one or more of the gallium compounds of the invention, or the gallium-containing composition can be administered subsequent to administration of the causative agent. As noted above, administration “with” another agent includes administration in the same or different composition, either simultaneously or sequentially. A gallium-containing composition is typically administered in an amount sufficient to achieve a serum gallium level of at least about 10 ng/mL, and typically reaches that level within 6 hours following administration.

When it is known that an individual will be exposed to a causative agent of an adverse liver condition, the individual can be treated with a gallium-containing composition in accordance. with methods of the invention in order to prevent or mitigate the damage that might be caused by the causative agent. Examples of such agents include, but are not limited to, natural or manufactured toxins, an environmental pollutant such as a pesticide, a chemical compound used in manufacturing, and an organic solvent. In one embodiment, the invention provides a method for mitigating potential liver damage resulting from exposure of an individual to a toxic substance, comprising administering a unit dose of a gallium-containing composition before, during, or subsequent to exposure of the individual to the toxic substance, wherein the unit dose comprises an amount of the gallium-containing composition sufficient to provide a prophylactically effective serum gallium level.

In one embodiment, the invention provides a method for mitigating potential liver damage resulting from administration of a pharmacologically active agent to an individual, comprising administering a unit dose of a gallium-containing composition before, during, or subsequent to administration of the pharmacologically active agent to the individual, wherein the unit dose comprises an amount of the gallium-containing compositing sufficient to provide a prophylactically effective serum gallium level. As used herein, “pharmacologically active agent” refers to a potentially therapeutically beneficial substance administered to or ingested by an individual with potentially hepatotoxic consequences. The pharmacologically active agent may be co-administered (simultaneously in separate formulations or in combination in a single formulation) with the gallium-containing composition, or may be administered sequentially at different times in separate formulations (i.e., within the context of different dosage regimens). Non-limiting examples of hepatotoxic prescription drugs that could be delivered with gallium include, but are not limited to, anti-inflammatory agents, lipid-lowering agents, immunosuppressant agents, antidiabetic agents, antibiotics, antifungal agents, retinoids, anticonvulsant agents, psychotropic agents, hormones, and combinations thereof. Specific examples include NSAIDs such as acetaminophen, statins, nicotinic acid, acarbose, pioglitazone, cyclosporine, sulfonylureas, amoxicillin, clarithromycin, erythromycin, tetracycline, trolendomycin, isoniazid, nitrofurantoin, fluconazole, fluoxetine, itraconazole, ketoconazole, etretinate, phenytoin, valproic acid, bupropion, chlorpromazine, tricyclic antidepressants, tamoxifen, testosterone, halothane, methotrexate, and combinations thereof.

Likewise, gallium can be administered prior to, concurrent with, or subsequent to radiation for the treatment of a cancer. In one embodiment, the invention provides a method for mitigating potential liver damage resulting from administration of radiation therapy to an individual, comprising administering a unit dose of a gallium-containing composition before, during, or subsequent to administration of radiation therapy to the individual, wherein the unit dose comprises an amount of the gallium-containing composition sufficient to provide a prophylactically effective serum gallium level.

Drug Delivery Systems

The compounds of the invention can be delivered transdermally or transmucosally, as discussed above. To facilitate this delivery, the compounds can be made available in a delivery system comprising a drug reservoir, a backing layer, and a means for affixing the system to the skin. The drug reservoir contains gallium in the form of a coordination complex of gallium (III), a salt of gallium (III), or protein-bound gallium (III), combined with a carrier suitable for transdermal or transmucosal drug delivery. Optionally, the drug reservoir is composed of a skin contact adhesive material suitable for affixing the system to the skin. Various transdermal drug delivery systems are known in the art, and can be combined with the compounds of the invention to enable practicing of the methods of the invention.

Pharmaceutical Compositions

The invention provides a pharmaceutical composition for treatment or mitigation of an adverse condition of the liver as described herein, comprising an amount of a gallium-containing composition as described herein sufficient to result in a therapeutically or prophylactically effective serum level, and a therapeutically effective amount of a second active agent indicated for treatment of the adverse liver condition.

In one embodiment, the adverse liver condition is caused by hepatitis, and the second active agent is an interferon, a nucleoside agent, or a combination thereof.

Kits

Kits are provided for use in methods of the invention for treatment or prevention of an adverse liver condition. The kits include a pharmaceutical composition for use in a method of the invention, for example, including at least one unit dose of a gallium-containing composition, and instructions providing information to a health care provider or patient regarding such usage. Instructions may be provided in printed form or in the form of an electronic medium such as a floppy disc, CD, or DVD, or in the form of a website address where such instructions may be obtained.

Suitable packaging is provided. As used herein, “packaging” refers to a solid matrix or material customarily used in a system and capable of holding within fixed limits a gallium-containing composition suitable for administration to an individual. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

In some embodiments, such kits can contain dosage forms, e.g., separately sealed, individually removable unit dosage forms packaged in a container, wherein each unit dosage form comprises (a) a pharmaceutical composition containing (i) a unit dosage of a gallium-containing composition as described herein, and (ii) a pharmaceutically acceptable carrier, wherein the unit dosage is effective to provide a therapeutically or prophylactically effective serum gallium level, for example, of at least 10 ng/mL, preferably within six hours following administration of the composition to a mammalian individual; and (b) instructions describing administration of the dosage forms in a manner effective to treat an adverse condition of the liver.

In some embodiments, the gallium-containing composition in the kit is in an orally active form, the pharmaceutically acceptable carrier is suitable for oral drug delivery, and the instructions describe oral administration of the dosage forms in a manner effective to treat an adverse condition of the liver.

It is to be understood that while the invention has been described in conjunction with the preferred specific embodiments thereof, that the foregoing description as well as the examples that follow are intended to illustrate and not limit the scope of the invention. Other aspects, advantages, and modifications within the scope of the invention will be apparent to those skilled in the art to which the invention pertains.

The following examples are intended to illustrate but not limit the invention.

### EXAMPLES

Unless noted otherwise, materials were obtained from commercially available sources and used without further purification.

### Example 1

A preclinical animal model was used to test the efficacy of oral gallium maltolate in treating hepatomegaly associated with adjuvant-induced acute arthritis. Male Lewis rats were used in the study. The study design was described in detail by Bendele et al. (1999) Toxicologic Pathology 27(1):134-142 and by Bendele (2001) J. Musculoskel. Neuron. Interact. 1(4):377-385.

Materials and Methods

Male Lewis rats (7 per group for gallium maltolate, 4 per group for normal controls and dexamethasone-treated controls) were injected with 100 μL of Freund's complete adjuvant/lipoidal amine (FCA/LA) subcutaneously at the base of the tail on study day 0 under anesthesia. There is a rapid onset (7 d) of arthritic symptoms in this model, including ankle inflammation, liver and spleen hypertrophy, bone resorption, and mild cartilage destruction.

Prophylactic treatment was initiated seven days prior to adjuvant injection by dosing with control vehicle or gallium maltolate (100 or 300 mg/kg gallium maltolate, containing 16 or 47 mg/kg gallium, respectively, in suspension with 1% carboxymethyl cellulose) seven days prior to adjuvant injection. Dosing was by daily oral gavage until termination. The dexamethasone-treated control animals were injected with a daily oral dose of dexamethasone (0.1 mg/kg). Body weights were measured regularly during the course of the study to track the effect of the drugs on the weight loss induced by the developing adjuvant disease, and dose volumes were adjusted accordingly. Prior to the onset of swelling, but after the establishment of systemic disease (about 7 days after adjuvant injection), caliper measurements were made of ankle joints. Ankles were measured every day until 14 days post-adjuvant injection when the rats were anesthetized and sacrificed. Serum was harvested one hour after final dosing for gallium quantitation. Hind paws, liver, and spleen were weighed, fixed, and processed for histopathologic evaluation. Adjuvant arthritic ankles were given scores of 0-5 (0=normal; 5=severe) for inflammation and bone resorption. Splenic changes of inflammation, increased extramedullary hematopoiesis, and lymphoid atrophy were scored 0-5 using criteria similar to those used for scoring of inflammation. The primary endpoint was hepatomegaly, as determined by liver weight and histopathology.

Results

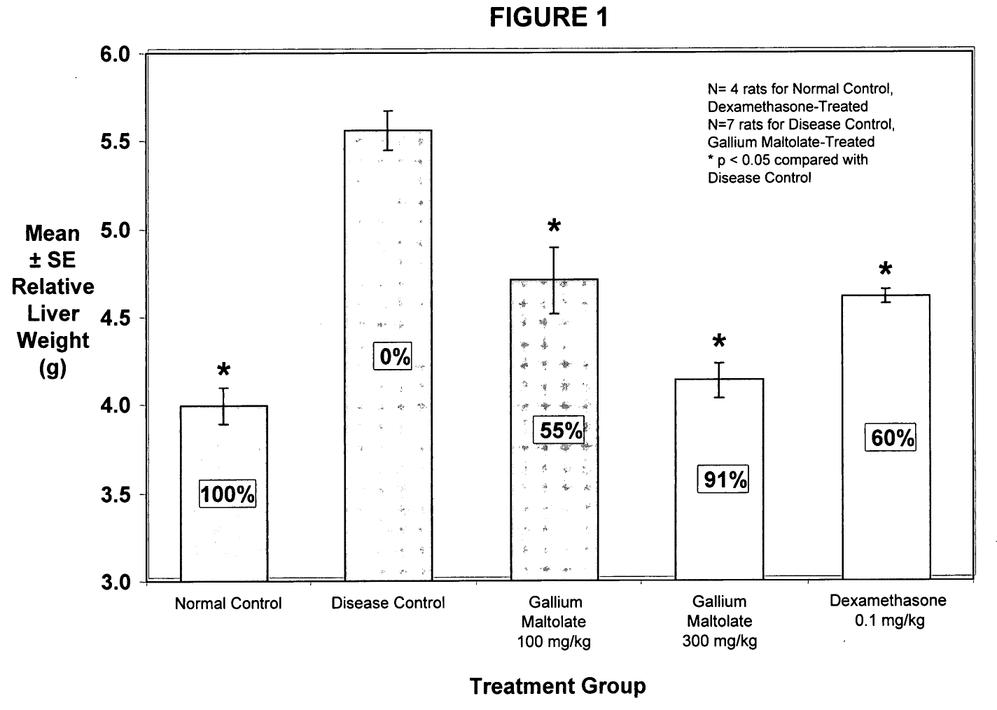

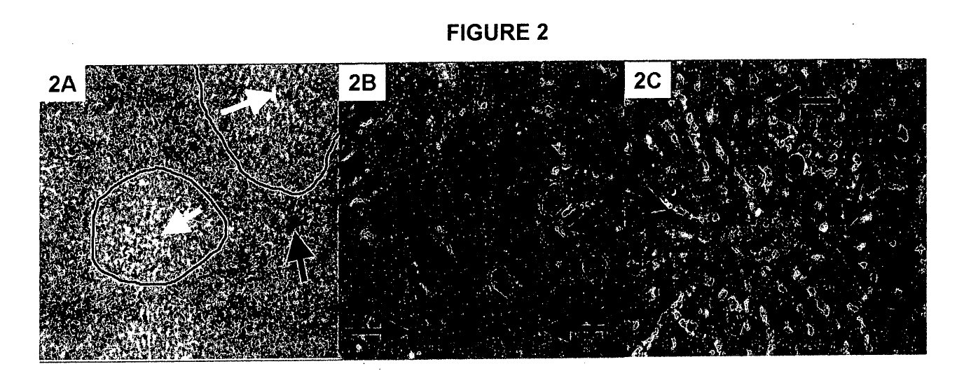

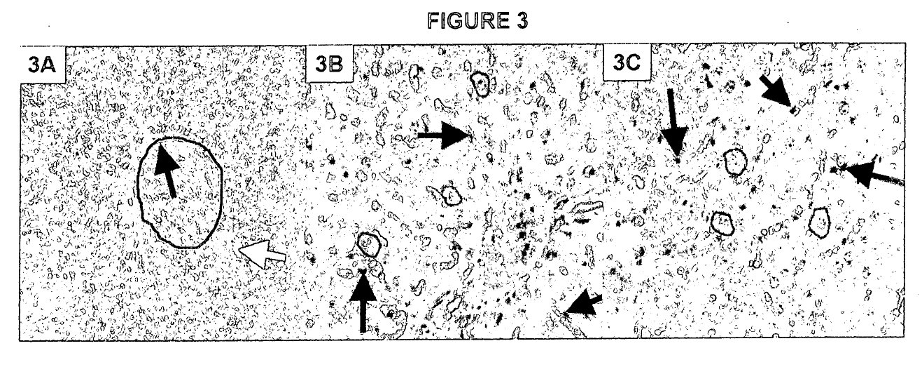

Daily oral dosing of gallium maltolate for 14 days in Lewis rats, at 100 or 300 mg/kg, produced no signs of toxicity, and serum gallium levels attained were dose-dependent. A marked reduction in liver and spleen hypertrophy at both doses indicated protection of the liver and spleen. Histopathological examination of the livers from gallium treated and untreated adjuvant arthritic rats showed that gallium treatment inhibited the development of liver pathology. The results are shown in FIGS. 1-3.

In summary, in the acute model for adjuvant-induced arthritis, oral gallium delivered as gallium maltolate was safe with no signs of toxicity observed after 14 days of daily administration. Significant dose-dependent protection from adjuvant-induced hepatomegaly was observed.

### Example 2

A human patient, prior to exposure to a liver-damaging agent, is treated with gallium in the form of gallium maltolate combined with the appropriate standard excipients and/or fillers, in tablet or capsule form. Sufficient gallium maltolate is administered to obtain at least 10 ng/mL gallium in the patient's serum. The amount of gallium maltolate plus excipients necessary to reach this level is dependent on the formulation used and the size of the patient. The patient continues to take the oral medication in an amount to build the level of serum gallium and to maintain the level of at least 10 ng/mL serum.

After a period of up to 72 hours, or when the gallium serum concentration reaches the desired level, the patient is exposed to the liver-damaging agent.

The serum levels of liver enzymes are monitored at the start of the gallium treatment and every 12 hours thereafter. In particular, levels of SDOT and SGOT are measured. It is expected that levels of these enzymes do not rise to any significant extent, indicating lack of damage to the patient's liver. Gallium maltolate administration is discontinued after risk of liver damage is terminated.

### Example 3

Concanavalin A (Con A) is a powerful mitogen for and activator of T lymphocytes and its intravenous administration to mice initiates an acute immune-mediated hepatitis. The effect of preemptive gallium maltolate administration on the development of acute liver injury was assessed in vivo.

Liver injury was induced by injecting 6-8 week old Balb/c male mice (weight 25 g) with Con A, 25 mg/kg in 250 μl of phosphate-buffered saline via the tail vein. The test animals were divided into three groups of 2-8 mice each:
(a) Con A treatment
(b) Vehicle (1% carboxymethyl cellulose solution in water) per oral gavage for 4 days prior to Con A treatment
(c) 150 mg/kg gallium maltolate per oral gavage for 4 days prior to Con A treatment

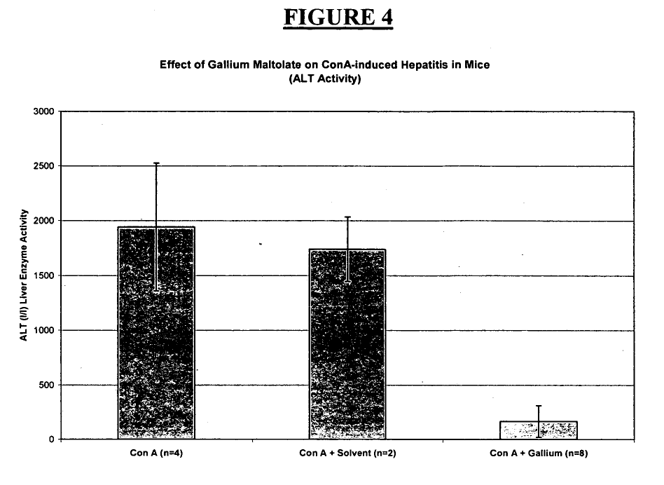

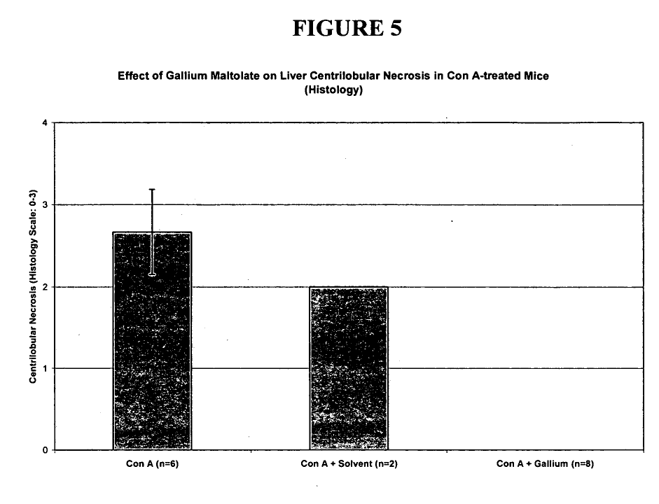

The results are shown in FIGS. 4 and 5. For determination of serum alanine aminotransferase (ALT) levels, blood was drawn from different groups of mice 16 hours after administering Con A and measured by a commercially available enzyme assay. ALT (liver cell lyase) levels were greatly reduced in mice administered gallium maltolate prior to ConA treatment versus mice in the other two test groups. (FIG. 4). Histological assessment of centrilobular necrosis of the liver was rated for severity on a scale of 0 to 3. (FIG. 5) No necrosis was observed in mice administered gallium maltolate prior to Con A treatment, whereas necrosis was observed in mice in the other two test groups.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes and to the same extent as if each individual publication, patent, or patent application were specifically and individually indicated to be so incorporated by reference.

We claim:

1. A method for treating an adverse condition of the liver in an individual in need thereof, comprising administering to the individual a unit dose of a gallium-containing composition, wherein said unit dose comprises an amount of said gallium-containing composition sufficient to provide a therapeutically effective serum gallium level.

2. The method of claim 1, wherein said therapeutically effective serum gallium level is at least about 10 ng/mL.

3. The method of claim 1, wherein said therapeutically effective serum gallium level is at least about 100 ng/mL.

4. The method of claim 1, wherein said therapeutically effective serum gallium level is at least about 500 ng/mL.

5. The method of claim 1, wherein said method comprises administering said gallium-containing composition to a human in an amount totaling about 2 mg/kg to about 550 mg/kg gallium per day.

6. The method of claim 5, wherein said gallium-containing composition is administered as a single dose per day.

7. The method of claim 5, wherein said gallium-containing composition is administered as multiple doses per day.

8. The method of claim 1, wherein said gallium-containing composition comprises a coordination complex of gallium(III), a salt of gallium (III), an inorganic compound of gallium (III), or protein-bound gallium (III).

9. The method of claim 1, wherein said gallium-containing composition comprises a coordination complex in the form of a neutral 3:1 (hydroxypyrone:gallium) complex in which each hydroxypyrone molecule is either unsubstituted or substituted with one, two, or three C₁-C₆ alkyl substituents.

10. The method of claim 9, wherein each hydroxypyrone molecule is selected from the group consisting of 3-hydroxy-4-pyrone, 3-hydroxy-2-methyl-4-pyrone, 3-hydroxy-2-ethyl-4-pyrone, and 3-hydroxy-6-methyl-4-pyrone.

11. The method of claim 10, wherein each hydroxypyrone molecule is 3-hydroxy-2-methyl-4-pyrone.

12. The method of claim 10, wherein each hydroxypyrone molecule is 3-hydroxy-2-ethyl-4-pyrone.

13. The method of claim 9, wherein said gallium-containing composition is administered orally.

14. The method of claim 1, wherein said therapeutically effective serum level is achieved within about 1 hour to about 12 hours after administration of the unit dose.

15. The method of claim 1, wherein the adverse condition comprises hypertrophy of the liver.

16. The method of claim 1, wherein the adverse liver condition is caused by alcohol use, a hepatotoxic medication, radiation, exposure to a toxic substance, or traumatic injury to the liver.

17. The method of claim 16, wherein the adverse liver condition is caused by a hepatotoxic medication selected from the group consisting of anti-inflammatory agents, lipid-lowering agents, immunosuppressant agents, antidiabetic agents, antibiotics, antifingal agents, retinoids, anticonvulsant agents, psychotropic agents, and hormones, and combinations thereof.

18. The method of claim 16, wherein the adverse liver condition is caused by exposure to a toxic substance selected from the group consisting of an environmental pollutant, a halide-hydrocarbon, petroleum, a petroleum byproduct, a pesticide, a chemical compound used in manufacturing, an organic solvent, and a pyrrolizidine alkaloid.

19. The method of claim 1, wherein the adverse liver condition comprises a liver disease selected from steatosis, alcoholic liver disease, primary biliary cirrhosis, hemochromatosis, Wilson's disease, a cystic disease, an inflammatory liver disease, hepatitis, and primary sclerosing cholangitis.

20. The method of claim 1, wherein said gallium-containing composition is administered in combination with a second active agent indicated for treatment of the adverse liver condition.

21. The method of claim 20, wherein the adverse liver condition is hepatitis and the second active agent is an interferon, a nucleoside agent, or a combination thereof.

22. A method for mitigating potential liver damage resulting from administration of a pharmacologically active agent to an individual, comprising administering a unit dose of a gallium-containing composition before, during, or subsequent to administration of the pharmacologically active agent to the individual, wherein said unit dose comprises an amount of said gallium-containing composition sufficient to provide a prophylactically effective serum gallium level.

23. The method of claim 22, wherein said prophylactically effective serum gallium level is at least about 10 ng/mL.

24. The method of claim 22, wherein said prophylactically effective serum gallium level is at least about 100 ng/mL.

25. The method of claim 22, wherein said prophylactically effective serum gallium level is at least about 500 ng/mL.

26. The method of claim 22, wherein the pharmacologically active agent and the gallium-containing composition are administered simultaneously.

27. The method of claim 26, wherein the pharmacologically active agent and the gallium-containing composition are administered in a single formulation.

28. The method of claim 22, wherein the pharmacologically active agent and the gallium-containing composition are administered sequentially.

29. The method of claim 28, wherein the pharmacologically active agent and the gallium-containing composition are administered within the context of different dosage regimens.

30. The method of claim 22, wherein the pharmacologically active agent is selected from the group consisting of anti-inflammatory agents, lipid-lowering agents, immunosuppressant agents, antidiabetic agents, antibiotics, antifungal agents, retinoids, anticonvulsant agents, psychotropic agents, hormones, and combinations thereof.

31. A method for mitigating potential liver damage resulting from radiation therapy to an individual, comprising administering a unit dose of a gallium-containing composition before, during, or subsequent to administration of radiation therapy to the individual, wherein said unit dose comprises an amount of said gallium-containing composition sufficient to provide a prophylactically effective serum gallium level.

32. The method of claim 31, wherein said prophylactically effective serum gallium level is at least about 10 ng/mL.

33. The method of claim 31, wherein said prophylactically effective serum gallium level is at least about 100 ng/mL.

34. The method of claim 31, wherein said prophylactically effective serum gallium level is at least about 500 ng/mL.

35. A method for mitigating potential liver damage resulting from exposure of an individual to a toxic substance, comprising administering a unit dose of a gallium-containing composition before, during, or subsequent to exposure of the individual to the toxic substance, wherein said unit dose comprises an amount of said gallium-containing composition sufficient to provide a prophylactically effective serum gallium level.

36. The method of claim 35, wherein said prophylactically effective serum gallium level is at least about 10 ng/mL.

37. The method of claim 35, wherein said prophylactically effective serum gallium level is at least about 100 ng/mL.

38. The method of claim 35, wherein said prophylactically effective serum gallium level is at least about 500 ng/mL.

39. The method of claim 35, wherein said toxic substance is selected from an environmental pollutant, a halide-hydrocarbon, petroleum, a petroleum byproduct, a pesticide, a chemical compound used in manufacturing, an organic solvent, and a pyrrolizidine alkaloid.

40. A pharmaceutical composition for treatment or mitigation of an adverse condition of the liver, the composition comprising: (a) an amount of a gallium-containing composition sufficient to provide a therapeutically effective serum gallium level; and (b) a therapeutically effective amount of a second active agent indicated for treatment of the adverse condition.

41. The pharmaceutical composition of claim 40, wherein the therapeutically effective serum gallium level is at least about 10 ng/mL.

42. The pharmaceutical composition of claim 40, wherein the therapeutically effective serum gallium level is at least about 100 ng/mL.

43. The pharmaceutical composition of claim 40, wherein the therapeutically effective serum gallium level is at least about 500 ng/mL.

44. The pharmaceutical composition of claim 40, wherein the adverse liver condition is hepatitis and the second active agent is an interferon, a nucleoside agent, or a combination thereof.

45. The pharmaceutical composition of claim 40, wherein said gallium-containing composition comprises a coordination complex of gallium (III), a salt of gallium (III), an inorganic compound of gallium (III), or protein-bound gallium (III).

46. The pharmaceutical composition of claim 40, wherein said gallium-containing composition comprises a coordination complex in the form of a neutral 3:1 (hydroxypyrone:gallium) complex in which each hydroxypyrone molecule is either unsubstituted or substituted with one, two, or three C₁-C₆ substituents.

47. The pharmaceutical composition of claim 46, wherein each hydroxypyrone molecule is selected from the group consisting of 3-hydroxy-4-pyrone, 3-hydroxy-2-methyl-4-pyrone, 3-hydroxy-2-ethyl-4-pyrone, and 3-hydroxy-6-methyl-4-pyrone.

48. The pharmaceutical composition of claim 46, wherein each hydroxypyrone molecule is 3-hydroxy-2-methyl-4-pyrone.

49. The pharmaceutical composition of claim 46, wherein each hydroxypyrone molecule is 3-hydroxy-2-ethyl-4-pyrone.

50. The pharmaceutical composition of claim 40, wherein the composition is formulated for parenteral administration.

51. The pharmaceutical composition of claim 40, wherein the composition is formulated for oral administration and the composition comprises an oral dosage form.

52. The pharmaceutical composition of claim 50, wherein the composition is formulated for transdermal or transmucosal administration.

53. A transdermal delivery system comprising a drug reservoir comprising the composition of claim 42.

54. A kit for treatment or mitigation of an adverse condition of the liver comprising (a) at least one unit dose of a gallium-containing composition, wherein the unit dose comprises an amount of the gallium-containing composition sufficient to provide a therapeutically or prophylactically effective serum gallium level following administration of the composition to an individual; and (b) instructions for use of the gallium-containing composition to treat or mitigate the adverse condition of the liver.

55. The kit of claim 54, wherein the gallium-containing composition is formulated for oral administration and the unit dose is in the form of an oral dosage form.

56. The kit of claim 55, wherein the gallium-containing composition comprises a coordination complex in the form of a neutral 3:1 (hydroxypyrone:gallium) complex in which each hydroxypyrone molecule is either unsubstituted or substituted with one, two, or three C₁-C₆ alkyl substituents.

57. The kit of claim 56, wherein each hydroxypyrone molecule is selected from the group consisting of 3-hydroxy-4-pyrone, 3-hydroxy-2-methyl-4-pyrone, 3-hydroxy-2-ethyl-4-pyrone, and 3-hydroxy-6-methyl-4-pyrone.

58. The method of claim 57, wherein each hydroxypyrone molecule is 3-hydroxy-2-methyl-4-pyrone.

59. The method of claim 57, wherein each hydroxypyrone molecule is 3-hydroxy-2-ethyl-4-pyrone.
